(19) United States
(12) Patent Application Publication
(10) Pub. No.: US 2014/0170210 A1
(43) Pub. Date: Jun. 19, 2014

(54) METHODS FOR TREATING IMMUNOSUPPRESSION

(71) Applicants: Ta-Yuan Chang, Etna, NH (US); Catherine C.Y. Chang, Etna, NH (US); Paul Li-Hao Huang, West Lebanon, NH (US)

(72) Inventors: Ta-Yuan Chang, Etna, NH (US); Catherine C.Y. Chang, Etna, NH (US); Paul Li-Hao Huang, West Lebanon, NH (US)

(73) Assignee: TRUSTEES OF DARTMOUTH COLLEGE, Hanover, NH (US)

(21) Appl. No.: 14/236,629

(22) PCT Filed: Aug. 2, 2012
(86) PCT No.: PCT/US12/49290

§ 371 Date: Mar. 4, 2014

Related U.S. Application Data
(60) Provisional application No. 61/514,699, filed on Aug. 3, 2011.

(51) Int. Cl. C12N 15/113; A61K 31/713; A61K 45/06

(52) U.S. Cl. CPC C12N 15/1137; A61K 45/06; A61K 31/713
USPC 424/450; 514/44 A

(57) ABSTRACT

The present invention includes methods for increasing white blood cell counts through administration of inhibitors of acyl-coenzyme A:cholesterol acyltransferase 1 (ACAT1). ACAT inhibitors are used to treat symptoms of immunosuppression, either alone or in combination with other treatments.

### INTRODUCTION

This application claims priority to U.S. Patent Application No. 61/514,699, filed Aug. 3, 2011, the content of which is incorporated herein by reference in its entirety.

This invention was made with government support under HL 060306 awarded by National Institutes of Health. The government has certain rights in the invention.

### BACKGROUND OF THE INVENTION

A low white blood cell count is often linked to problems with the bone marrow and the inability to make enough white blood cells. A poorly controlled or untreated low white blood cell count can be serious and even life threatening due to increased vulnerability to infectious diseases, i.e., immunosuppression. Chemotherapy and high dose radiation therapy can cause severe deficiency in white blood cell count. In addition, many diseases and disorders can cause a low white blood cell count, including-AIDS, aplastic anemia (condition in which the bone marrow makes insufficient blood cells), bone marrow disease (myelodysplastic syndromes), leukemia, liver disease (hepatitis, cirrhosis, and liver failure), overactive spleen that destroys white blood cells, rheumatoid arthritis (chronic autoimmune disease characterized by joint inflammation), systemic lupus erythematosus (disorder in which the body attacks its own healthy cells and tissues), viral infection that affects bone marrow function, vitamin deficiency, and widespread infection that depletes white blood cells. In addition, there are drugs that reduce the number of white blood cells as a part of the mechanism of action or as an adverse effect (e.g., antibiotics, anticonvulsants, antihistamines, antithyroid drugs, arsenic, barbiturates).

For treating patients with low white blood cell count, typically, various biologic agents are used to boost the immune response to raise cell numbers. These agents include interferon (IFN), interleukins (ILs), granulocyte colony stimulating factors (GCSF), and monoclonal antibodies. Due to differences in patient populations in response to various drugs and their underlying mechanisms of action, there is always a need for agents that can be used to increase white cell counts in patients through recruitment of new biological pathways.
Acyl-coenzyme A:cholesterol acyltransferases (ACATs) are membrane-bound proteins that utilize long-chain fatty acyl-coenzyme A and cholesterol as substrates to form cholesteryl esters, giving them an important role in cellular cholesterol homeostasis in various tissues. In mammals, two isoenzymes, ACAT1 and ACAT2, are encoded by two different genes (Buhman et al. 2000. Biochim. Biophys. Acta. 1529:142-154; Rudel et al. 2001. Curr. Opin. Lipidol. 12:121-127). In monocytes/macrophages and most other cell types examined, ACAT1 is the major isoenzyme (Meiner et al. 1996. PNAS USA 93:14041-14046; Miyazaki et al. 1998. Arterioscl. Thromb. Vasc. Biol. 18:1568-1574; Farese, R. V. 1998. Curr. Opin. Lipidol. 9:119-124). ACAT2 is the major isoenzyme in mouse liver and intestine, whereas both ACAT1 and ACAT2 are present in human liver (Buhman et al. 2000. Nat. Med. 6:1341-1347; Lee et al. 2000. J. Lipid Res. 41:1991-2001; Parino et al. 2004. Circulation 110:2017-2023; Chang et al. 2009. Am. J. Physiol. 297:E1-E9). Both enzymes are potential drug targets for treating patients with atherosclerosis (Chang et al. 2009. Am. J. Physiol. 297:E1-E9; Rudel and Farese. 2006. NEJM 354:2616-2617). ACAT1 is also a potential target for treating patients with Alzheimer's disease (Bryleva et al. 2010. PNAS USA 107:3081-3086).

In animal studies, Fazio and colleagues prepared and transplanted bone marrow from donor Acat1+/+ or Acat1 knockout (Acat1−/−) mice into recipient irradiated low density lipoprotein receptor knockout (Ldlr−/−) or apolipoprotein E knockout (Apoe−/−) mice, and showed that atherosclerotic lesions were enlarged when donor bone marrow from the total Acat1−/− mouse was employed (Fazio et al. 2001. J. Clin. Invest. 107:163-171; Su et al. 2005. Circulation 111:2373-2381). These authors concluded that the deleterious effect on progression of atherosclerosis in the knockout mice was caused by a lack of Acat1 in macrophages. However, there are alternative explanations for their findings. For example, bone marrow contains hematopoietic stem cells (HSC), which give rise to all blood cell types in the myeloid lineage (e.g., monocytes/macrophages, neutrophils, and dendritic cells) and the lymphoid lineage (e.g., T cells and B cells). In addition to affecting monocytes/macrophages, total lack of Acat1 activity in Acat1−/− mice may affect the functionality of HSCs within the bone marrow. In fact, genetic inactivation of lysosomal acid lipase, a key enzyme that hydrolyzes cholesteryl esters to unesterified cholesterol in the endo/lysosomes, increases HSC proliferation in the bone marrow and results in aberrant growth and differentiation of myeloid progenitor cells (Qu et al. 2010. Am. J. Pathol. 176:2394-2404). Genetic inactivation of ABCA1 and ABCG1, two key proteins involved in cellular cholesterol efflux, also causes increased proliferation of HSC in the bone marrow and resulted in increased leukocytosis (Yvan-Charvet et al. 2010. Science 328:1689-1693). These studies show that bone marrow function can be affected by alterations in normal cholesterol homeostasis in cells. Inhibiting lysosomal acid lipase has no practical therapeutic value, because lacking lysosomal acid lipase can lead to a serious disease called Wolman Disease in humans. Inhibiting ABCA1 has no practical therapeutic value, either, because lacking ABCA1 leads to a serious disease called Tangier Disease in humans.

It has now been found that inactivation of ACAT1 function in bone marrow HSCs can lead to an increase in white blood cell count in blood, an effect that would be useful for treatment of immunosuppression associated with disease, or with drug treatment of disease (i.e., cancer chemotherapy), or with any condition wherein the activity of white blood cells is needed to maintain health of an individual. Unlike deficiency in lysosomal acid lipase or deficiency in ABCA1, ACAT1 deficiency has very minor adverse phenotypes in mouse (Meiner et al. 1996. PNAS USA 93:14041-14044). In addition, two clinically safe orally available small molecule ACAT inhibitors are known in the art; i.e., Avasimibe, described in Tardif, et al. 2004. Circulation 110:3372-3377; and Pactimibe, described in Nissen et al. 2006. NEJM 354:1253-1263.

### BRIEF DESCRIPTION OF THE DRAWINGS

FIG. 1 shows that Acat1−/− displays leukocytosis. Peripheral blood leukocytes were analyzed by flow cytometry. FIG. 1A. WT and Acat1−/− mice at 8 weeks of age with comparable numbers in males and females were used. Each circle represents result from one animal. Markers used: CD11b for monocytes, Gr1 for neutrophils, CD19 for B cells, and CD3e for T cells. FIGS. 1B and 1C. Recipient WT mice (FIG. 1B) or Acat1−/− mice (FIG. 1C) transplanted with either WT or Acat1−/− BM as indicated. Leukocytes were measured 7, 9, or 11 week post BMT as indicated. Results are means±SEM, using 21 WT mice and 13 Acat1−/− mice in 2 separate experiments. *P<0.05, ***P<0.001 vs. WT mice.

FIG. 2 results of flow cytometry examining white blood cell populations 7 to 11 weeks after bone marrow transplant. Experiments employed four or more mice per group per experiment, and involved three experiments. Results reported are means±SEM. *P<0.05, **P<0.01, ***P<0.001.

FIG. 3 shows that Acat1−/− increases myeloid cells and B Cells in BM. BM leukocytes were analyzed by flow cytometry. FIG. 3A. BM myeloid cells (CD11b⁺Gr1⁺). FIG. 3B. B cells (CD19⁺). Results were reported as total cell number (left panels) and percentage in BM (right panels). Results are means±SEM. For total BM cells, 24 WT mice and Acat1−/− mice in 5 separate experiments; for myeloid cells, 13 WT mice and 13 Acat1−/− mice in 3 separate experiments; for B cells, 24 WT mice and 24 Acat1−/− mice in 5 separate experiments. **P<0.01 vs. WT mice.

FIG. 4 shows that Acat1−/− increases proliferation of hematopoietic progenitor cells in BM. FIG. 4A, LSK, CMP, GMP, MEP, and CLP cells in BM were quantified by flow cytometry; results were reported as total number (left panel) and as percentage within the BM (right panel). FIGS. 4B and 4C, Proliferation (FIG. 4B) and apoptosis (FIG. 4C) of various progenitors were quantified by BrdU (FIG. 4B) and Annexin V staining (FIG. 4C). Results are means±SEM. For FIG. 4A, 15 WT mice and 15 Acat1−/− mice in 4 separate experiments for LSK, CMP, MEP, and GMP determinations; 6 WT mice and 6 Acat1−/− mice in 2 separate experiments for CLP determinations. For FIGS. 4B and 4C, 8 WT mice and 8 Acat1−/− mice in 2 separate experiments. ***P<0.001, *P<0.05 vs. WT mice.

FIG. 5 shows that cultured Acat1−/− BM cells proliferate faster through activated IL3Rβ/p-ERK pathway. WT and Acat1−/− BM cells were cultured in IMDM media containing 10% FBS in the presence of indicated growth factors with or without specific inhibitors as indicated for 72 hours. Concentrations of growth factors used: 100 ng/mL for stem cell factor, 6 ng/mL for IL3; 2 ng/ml for GM-CSF. Concentration of inhibitors used: 10 μM for U0126; 1 μM for PP2. Proliferation was measured after a 3 hour ³H-thymidine pulse in intact cells. Results are means±SEM of duplicate cultures in 3 separate experiments. **P_{<}0.01, * P<0.05 vs. WT.

FIG. 6 shows that Acat1−/− LSK cells have higher IL3Rβ expression in vivo and in culture. Cell surface IL3Rβ expression was monitored by using flow cytometry. The graph depicts the expression in LSK cells and in total BM cells in vivo. Results are from 8 WT mice and 8 Acat1−/− mice in separate experiments. Results are means±SEM. *P_{<}0.05 vs. WT.

FIG. 7 shows that Acat1−/− affects B Cell progenitors. BM B progenitor cells in WT and Acat1−/− BMs were analyzed by flow cytometry. FIG. 7A. BM B cells (CD19⁺), Pro-B (CD43⁺CD19⁺) and Pre-B (CD43⁻CD19⁺IgM⁻) were quantitated as total cell number (left panel) and as percentage within the BM (right panel). FIG. 7B. Proliferation and apoptosis of B and B progenitor cells were quantified by BrdU (left panel) and Annexin V staining (right panel). FIG. 7C. Percentage of cell surface IL7Rα expression in B and B progenitor cells were measured by using flow cytometry. Results are means±SEM. For FIG. 7A. 24 WT mice and 24 Acat1−/− mice in 5 separate experiments. For FIG. 7B, 8 WT mice and 8 Acat1−/− mice in 2 separate experiments. For FIG. 7C, 6 WT mice and 6 Acat1−/− mice in 2 separate experiments. *2<0.05, **P<0.01 vs. WT mice.

FIG. 8 depicts analysis of blood leukocytes of 7 to 8 week old (age- and sex-matched) wild-type (WT), Acat1−/−, and Acat1−M/−M mice using flow cytometry. Each point represents a value from one mouse. The Acat1−M/−M mouse is a genetically engineered mouse that is specifically deficient in ACAT1 in macrophage only, but not deficient in ACAT1 in other cell types.

### SUMMARY OF THE INVENTION

The present invention is a specific enzyme-based method for increasing white blood cell count in a patient. The method of the invention involves administering to a patient an effective amount of an ACAT inhibitor in a pharmaceutically acceptable vehicle. The present invention is also a method for treating immunosuppression by administering to a patient diagnosed with low white blood cell count, or predicted/predisposed to have low white blood cell count immediately after standard radiation therapy or standard chemical therapy, an effective amount of an ACAT inhibitor in a pharmaceutically acceptable vehicle, so that the signs and/or symptoms of low white blood cell count are decreased, delayed, or prevented thereby treating immunosuppression. In some embodiments, the ACAT inhibitor inhibits both ACAT1 and ACAT2. In other embodiments, the ACAT inhibitor is a selective inhibitor of ACAT1. In accordance with this embodiment, the ACAT inhibitor has an IC₅₀ value for ACAT1 which is at least twice the corresponding IC₅₀ value for ACAT2. In particular embodiments, the selective inhibitor of ACAT1 is an siRNA or microRNA. ACAT inhibitors of the invention can be administered via a liposome or nanoparticle, can have an IC₅₀ value in the range of 1 nM to 100 μM, and can be administered in combination with another drug used to treat immunosuppression.

### DETAILED DESCRIPTION OF THE INVENTION

It has now been found that white blood cell counts in ACAT1 gene knockout mice (Acat1−/− mice) are significantly higher than white blood cell counts in wild-type mice (Acat1+/+ mice). It has further been shown in bone marrow transplantation experiments that donor bone marrow from Acat1−/− mice can stimulate production of white blood cells in recipient, wild-type mice. Bone marrow samples from the Acat1−/− mice also have been found to contain more common myeloid progenitor cells. Finally, it was found that in cultured bone marrow cells, treatment with interleukin 3 (IL3) resulted in an increased rate of proliferation of Acat1−/− bone marrow cells as compared to normal bone marrow cells. Taken together, these data demonstrate that inhibition of ACAT1 function (Acat1−/− phenotype) stimulates cell proliferation within HSC lineage, and causes a significant increase in white blood cell number. This new role for ACAT1 in HSC lineage indicates that agents that inhibit ACAT1 activity can be used therapeutically in patients with low white blood cell counts. The low white blood cell count may occur due to drug treatment, radiation treatment, or may be the result of certain diseases. As is understood by one of skill in the art, under these conditions, an increase in white blood cell count in blood is an effect that would be beneficial clinically.

Having demonstrated that Acat1 gene ablation was associated in vivo with significant changes in white blood cell count, the present invention provides the first evidence that ACAT inhibitors would be useful as treatment for diseases associated with immunosuppression, specifically decreased white blood cell count.

In the context of the present invention, “immunosuppression” is a condition associated with lower than normal white blood cell count, wherein a normal white blood cell count is defined by standard laboratory reference ranges that would be known by one of skill in the art. A “low” white blood cell count is defined in the context of the present invention as any number of white blood cells that is below the low end of a standard laboratory reference range. Such standard laboratory reference ranges for white blood cell count would include but not be limited to those identified in standard laboratory manuals such as Tietz Clinical Guide to Laboratory Tests (Wu, A. H. B. 2006. 4^{th} edition. Saunders Elsevier: St; Louis Mo.). A significant increase in white blood cell count as observed in the context of the present invention can also be referred to as “leukocytosis”, which is a condition wherein there is a higher than normal number of circulating white blood cells.

As a result, the present invention is a method for treating immunosuppression in humans by administering to a patient diagnosed with low white blood cell count an effective amount of an ACAT inhibitor in a pharmaceutically acceptable vehicle. In accordance with the methods of this invention, a subject or patient, suspected or predicted of having or diagnosed low white blood cell count is administered an effective amount of an agent that inhibits the activity of ACAT1 so that the signs and/or symptoms of low white blood cell count are decreased, delayed, or prevented thereby treating immunosuppression.

In some embodiments, the ACAT inhibitor inhibits ACAT1, but also to a certain degree ACAT2. In this respect, the ACAT1 inhibitor is a non-selective ACAT inhibitor that is capable of inhibiting both ACAT1 and ACAT2. Inhibitors that inhibit both isoforms of ACAT include, for example, Avasimibe and Pactimibe. Avasimibe, also known as CI-1011, is an oral ACAT inhibitor that is non-selective as it inhibits both ACAT1 and ACAT2 activity with approximately equal potency (IC₅₀ of 18.7 μM and 19.1 μM, respectively; Ikenoya et al. 2007. Atherosclerosis 191:290). It has been shown to be safe when administered to rats, dogs, and humans (Llaverías et al. 2003. Cardiovasc. Drug Rev. 21:33-50). In vitro studies in human macrophages have demonstrated that Avasimibe reduces foam cell formation in macrophages by enhancing free cholesterol efflux and inhibiting the uptake of modified low-density lipoprotein (LDL; Rodriguez and Usher. 2002. Atherosclerosis 161:45-54). Studies in animal models have suggested that Avasimibe treatment could contribute to increased plaque stability in atherosclerosis (as reviewed in Llaverías et al. 2003. Cardiovasc. Drug Rev. 21:33-50). In clinical studies in humans, Avasimibe has been administered in combination with a statin drug, but failed to demonstrate efficacy to further reduce progression of coronary atherosclerosis in patients (Tardif et al. 2004. Circulation 110:3372-3377). Avasimibe also has been tested for efficacy to treat Alzheimer's disease (Huttunen et al. 2009. FASEB J. 23:3819-3828). Administering Avasimibe to a mouse model for Alzheimer disease led to significant decreases in amyloid plaque load and a reduction in cognitive deficits normally manifested in these animals (Huttunen et al. 2009. FASEB J. 23:3819-3828). Pactimibe is from the same drug class as Avasimibe (Terasaka et al. 2007. Atherosclerosis 190:239-247). Like Avasimibe, Pactimibe also inhibits both ACAT1 and ACAT2 with approximately equal potency (Kitayama et al. 2006. Eur. J. Pharmacol. 540:121-130), and has been administered to humans in clinical trials. Pactimibe also lacked efficacy as a supplement to statin treatment in patients with high risk for atherosclerosis, or with familial hypercholesterolemia (Nissen et al. 2006. NEJM 354:1253-1263; Meuwese et al. 2009. JAMA 301:1131-1139). Additional non-selective ACAT inhibitors of use in the current method include, but are not limited to CP-113,818 (Gutter-Paier et al. 2004. Neuron 44:227-238) and CI-976 (Chang et al. 2000. J. Biol. Chem. 275:28083-28092).

In other embodiments of the present invention, the inhibitor is a selective inhibitor of ACAT1. As used herein, a “selective inhibitor of ACAT1” or “ACAT1-selective inhibitor” is any molecular species that is an inhibitor of the ACAT1 enzyme but which fails to inhibit, or inhibits to a substantially lesser degree ACAT2. Methods for assessing the selectively of ACAT1 inhibitors are known in the art and can be based upon any conventional assay including, but not limited to the determination of the half maximal (50%) inhibitory concentration (IC) of a substance (i.e., 50% IC, or IC₅₀), the binding affinity of the inhibitor (i.e., Kᵢ), and/or the half maximal effective concentration (EC₅₀) of the inhibitor for ACAT1 as compared to ACAT2. See, e.g., Lada, et al. (2004) J. Lipid Res. 45:378-386 and U.S. Pat. No. 5,968,749. ACAT1 and ACAT2 proteins that can be employed in such assays are well-known in the art and set forth, e.g., in GENBANK Accession Nos. NP_{—}000010 (human ACAT1) and NP_{—}005882 (human ACAT2). See also U.S. Pat. No. 5,834,283.

In particular embodiments, a “selective ACAT1 inhibitor” is an agent exhibiting an IC₅₀ value for ACAT1 that is at least twice or, more desirably, at least three, four, five, or six times higher than the corresponding IC₅₀ value for ACAT2. Most desirably, a selective inhibitor of ACAT1 has an IC₅₀ value for ACAT1 which is at least one order of magnitude or at least two orders of magnitude higher than the IC₅₀ value for ACAT2.

Selective inhibitors of ACAT1 activity have been described. For example, Ikenoya et al. (2007. Atherosclerosis 191:290-297) teach that K-604 has an IC₅₀ value of 0.45 μmol/L for human ACAT1 and 102.85 μmol/L for human ACAT2. As such K-604 is 229-fold more selective for ACAT1 than ACAT2. K604 has been used in vivo in laboratory animals and has been shown to be safe for use in vivo (Ikenoya et al. 2007. supra). In addition, diethyl pyrocarbonate has been shown to inhibit ACAT1 with 4-fold greater activity (IC₅₀=44 μM) compared to ACAT-2 (IC₅₀=170 μM) (Cho et al. 2003. Biochem. Biophys. Res. Comm. 309:864-872). Ohshiro et al. (2007. J. Antibiotics 60:43-51) teach selective inhibition of beauveriolides I (0.6 μM vs. 20 μM) and III (0.9 μM vs. >20 μM) for ACAT1 over ACAT2. In addition, beauveriolide analogues 258, 280, 274, 285, and 301 show ACAT1-selective inhibition with pIC₅₀ values in the range of 6 to 7 (Tomoda & Doi. 2008. Accounts Chem. Res. 41:32-39). Lada, et al. (2004. J. Lipid Res. 45:378-386) teach a Warner-Lambert compound (designated therein as Compound 1A), and derivatives thereof (designated Compounds 1B, 1C, and 1D), which inhibit ACAT1 more efficiently than ACAT2 with IC₅₀ values 66- to 187-fold lower for ACAT1 than for ACAT2. Moreover, Lee et al. 2004. Bioorg. Med. Chem. Lett. 14:3109-3112) teach methanol extracts of Saururus chinensis root that contain saucerneol B and manassantin B for inhibiting ACAT activity. Saucerneol B inhibited hACAT-1 and hACAT-2 with IC₅₀ values of 43.0 and 124.0 μM, respectively, whereas manassantin B inhibited hACAT-1 and hACAT-2 with IC₅₀ values of 82.0 μM and only 32% inhibition at 1 mM, respectively.

Desirably, ACAT inhibitors of the present invention have an IC₅₀ value in the range of 1 nM to 100 μM. More desirably, ACAT inhibitors of the invention have an IC₅₀ value less than 100 μM, 50 μM, 10 μM, or 1 μM. Most desirably, ACAT inhibitors of the invention have an IC₅₀ value in the nM range (e.g., 1 to 999 nM).

In addition to the above referenced ACAT inhibitors, it is contemplated that any conventional drug screening assay can be employed for identifying or selecting additional or more selective ACAT1 inhibitors or derivatives or analogs of known ACAT1 inhibitors. See, e.g., Lada et al. 2004. J. Lipid Res. 45:378-386. Such agents can be identified and obtained from libraries of compounds containing pure agents or collections of agent mixtures. Examples of pure agents include, but are not limited to, proteins, peptides, nucleic acids, oligonucleotides, carbohydrates, lipids, synthetic or semi-synthetic chemicals, and purified natural products. Examples of agent mixtures include, but are not limited to, extracts of prokaryotic or eukaryotic cells and tissues, as well as fermentation broths and cell or tissue culture supernates. In the case of agent mixtures, one may not only identify those crude mixtures that possess the desired activity, but also monitor purification of the active component from the mixture for characterization and development as a therapeutic drug. In particular, the mixture so identified may be sequentially fractionated by methods commonly known to those skilled in the art which may include, but are not limited to, precipitation, centrifugation, filtration, ultrafiltration, selective digestion, extraction, chromatography, electrophoresis or complex formation. Each resulting subfraction may be assayed for the desired activity using the original assay until a pure, biologically active agent is obtained.

Library screening can be performed in any format that allows rapid preparation and processing of multiple reactions such as in, for example, multi-well plates of the 96-well variety. Stock solutions of the agents as well as assay components are prepared manually and all subsequent pipetting, diluting, mixing, washing, incubating, sample readout and data collecting is done using commercially available robotic pipetting equipment, automated work stations, and analytical instruments for detecting the signal generated by the assay. Examples of such detectors include, but are not limited to, luminometers, spectrophotometers, calorimeters, and fluorimeters, and devices that measure the decay of radioisotopes. It is contemplated that any suitable ACAT enzymatic assay can be used in such screening assays.

As disclosed herein, there are a number of suitable molecules that selectively inhibit the activity of ACAT without modulating the expression of ACAT. However, in some embodiments of the present invention, an “ACAT inhibitor” inhibits the expression of ACAT1, thus achieving the purpose of inhibiting the ACAT1 enzyme activity. Molecules that can inhibit ACAT expression include, e.g., siRNA, antisense molecules, or ribozymes. In particular embodiments, the ACAT inhibitor selectively inhibits the expression of ACAT1, without modulating the expression of ACAT2. While some RNAi molecules have been shown to induce significant neurotoxicity in brain tissue (McBride, et al. (2008) Proc. Natl. Acad. Sci. USA 105:5868-5873), specific embodiments of this invention embrace one or more siRNA or microRNA molecules as the ACAT1-selective inhibitor. As is conventional in the art, miRNA or microRNA refer to 19-25 nucleotide non-coding RNAs derived from endogenous genes that act as post-transcriptional regulators of gene expression. They are processed from longer (ca 70-80 nucleotide) hairpin-like precursors termed pre-miRNAs by the RNAse III enzyme Dicer. MicroRNAs assemble in ribonucleoprotein complexes termed miRNPs and recognize their target sites by antisense complementarity thereby mediating down-regulation of their target genes. By way of illustration, target sequences for mouse ACAT1 microRNA molecules include, but are not limited to, those listed in Table 1.

TABLE 1

| | | SEQ ID |
|---|---|---|
| microRNA | ACAT1 Target Sequence | NO: |
| #52 | GGAGCTGAAGCCACTATTTAT | 1 |
| #53 | CTGTTTGAAGTGGACCACATCA | 2 |
| #54 | CCCGGTTCATTCTGATACTGGA | 3 |
| #55 | AACTACCCAAGGACTCCTACTGTA | 4 |

For example, pre-microRNAs (including sense, antisense and loop regions) that target microRNAs #54 and #55 were generated and shown to decrease mouse ACAT1 expression. These pre-microRNAs included 5′-TGC TGT CCA GTA TCA GAA TGA ACC GGG TTT TGG CCA CTG ACT GAC CCG GTT CAC TGA TAC TGG A-3′ (SEQ ID NO:5) and 5′-TGC TGT ACA GTA GGA GTC CTT GGG TAG TTT TGG CCA CTG ACT GAC TAC CCA AGC TCC TAC TGT A-3′ (SEQ ID NO:6).

Artificial microRNAs against human ACAT1 gene (e.g., GENBANK Accession No. NM_{—}000019) were also generated and shown to decrease human ACAT1 protein expression by 80% in human cells. Exemplary microRNA sequences targeting human ACAT1 include, but are not limited, those listed in Table 2.

TABLE 2

| MicroRNA Sequence (5′->3′) | SEQ ID NO: |
|---|---|
| CAUGAUCUUCCAGAUUGGAGUUCUA | 7 |
| UAGAACUCCAAUCUGGAAGAUCAUG | 8 |

In a similar manner, microRNA or siRNA against the ACAT1 gene in primates (e.g., GENBANK Accession No. XM_{—}508738, incorporated by reference) can be developed, and used to selectively inhibit the expression of primate ACAT1.

SiRNA or microRNA molecules which selectively inhibit the expression of ACAT1 or ACAT2 can be administered as naked molecules or via vectors (e.g., a plasmid or viral vector such as an adenoviral, lentiviral, retroviral, adeno-associated viral vector or the like) harboring nucleic acids encoding the siRNA or microRNA. Desirably, a vector used in accordance with the invention provides all the necessary control sequences to facilitate expression of the siRNA or microRNA. Such expression control sequences can include but are not limited to promoter sequences, enhancer sequences, etc. Such expression control sequences, vectors and the like are well-known and routinely employed by those skilled in the art. In particular embodiments, the siRNA or microRNA is delivered by a non-viral delivery method, e.g., liposome, nanoparticle, or liposome-siRNA-peptide complex (Pulford et al. 2010. PloS One 5:e11085.

Routine experimentation can be performed to demonstrate the efficacy of ACAT inhibitors in treating immunosuppression, specifically increasing white blood cell count. The first step is to examine the pharmacokinetics of an ACAT inhibitor, specifically its ability to distribute into the bone marrow, where HSCs originate, when administered systemically, the preferred route of administration for a human drug product. In the context of the present invention, systemic administration includes administration orally, subcutaneously, by intravenous injection, rectally, topically, or by inhalation. Next, the efficacy of an ACAT inhibitor is demonstrated first in an animal model of immunosuppression, and then in clinical studies. Progressing through each of these steps is the routine method used when developing a human drug.

When used in the methods of this invention, one or more ACAT inhibitors are administered to a subject in need of treatment in an amount that effectively reduces the expression or the activity of ACAT1. The level of ACAT1 inhibition could range anywhere from 30% to 100%, i.e., enough to cause leukocytosis in the treated subject. Subjects benefiting from treatment with an inhibitor of the invention include subjects confirmed as having low white blood cell count, subjects suspected of having low white blood cell count, or subjects predisposed to have low white blood cell count. In the context of this invention, a patient or subject can be any mammal including human, companion animals (e.g., dogs or cats), livestock (e.g., cows, sheep, pigs, or horses), or zoological animals (e.g., rabbits, monkeys). In particular embodiments, the subject is a human.

While certain embodiments of this invention embrace in vivo applications, in vitro use of inhibitors of the invention are also contemplated for examining the effects of ACAT inhibition on particular populations of white blood cells, and/or particular populations of hematopoietic stem cells of mouse or human origin.

When used in therapeutic applications, an ACAT inhibitor of the invention will have the therapeutic benefit of decreasing, reducing or ameliorating one or more signs or symptoms of immunosuppression, including, but not limited to low white blood cell count in the subject as compared to subjects not receiving treatment with the ACAT inhibitor.

For therapeutic use, ACAT inhibitors can be formulated with a pharmaceutically acceptable carrier at an appropriate dose. Such pharmaceutical compositions can be prepared by methods and contain carriers which are well-known in the art. A generally recognized compendium of such methods and ingredients is Remington: The Science and Practice of Pharmacy, Alfonso R. Gennaro, editor, 20th ed. Lippincott Williams & Wilkins: Philadelphia, Pa., 2000. A pharmaceutically acceptable carrier, composition or vehicle, such as a liquid or solid filler, diluent, excipient, or solvent encapsulating material, is involved in carrying or transporting the subject agent from one organ, or portion of the body, to another organ, or portion of the body. Each carrier must be acceptable in the sense of being compatible with the other ingredients of the formulation and not injurious to the patient.

Examples of materials which can serve as pharmaceutically acceptable carriers include sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Compositions of the present invention can be administered parenterally (for example, by intravenous, intraperitoneal, subcutaneous or intramuscular injection), topically, orally, intranasally, intravaginally, or rectally according to standard medical practices.

The selected dosage level of an ACAT inhibitor will depend upon a variety of factors including the activity of the particular agent of the present invention employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and other factors well-known in the medical arts.

It is also contemplated that ACAT inhibitors can be used in the methods of the present invention either alone or in combination with other agents commonly used to treat immunosuppression or to boost white blood cell counts in patients. Such agents would include but not be limited to interferon (IFN), interleukins (ILs), granulocyte colony stimulating factors (GCSF), and monoclonal antibodies. One of skill in the art would choose which agents to use in combination based on their clinical experience with such drugs, using doses approved for use in humans per the labeling for the drug products as marketed.

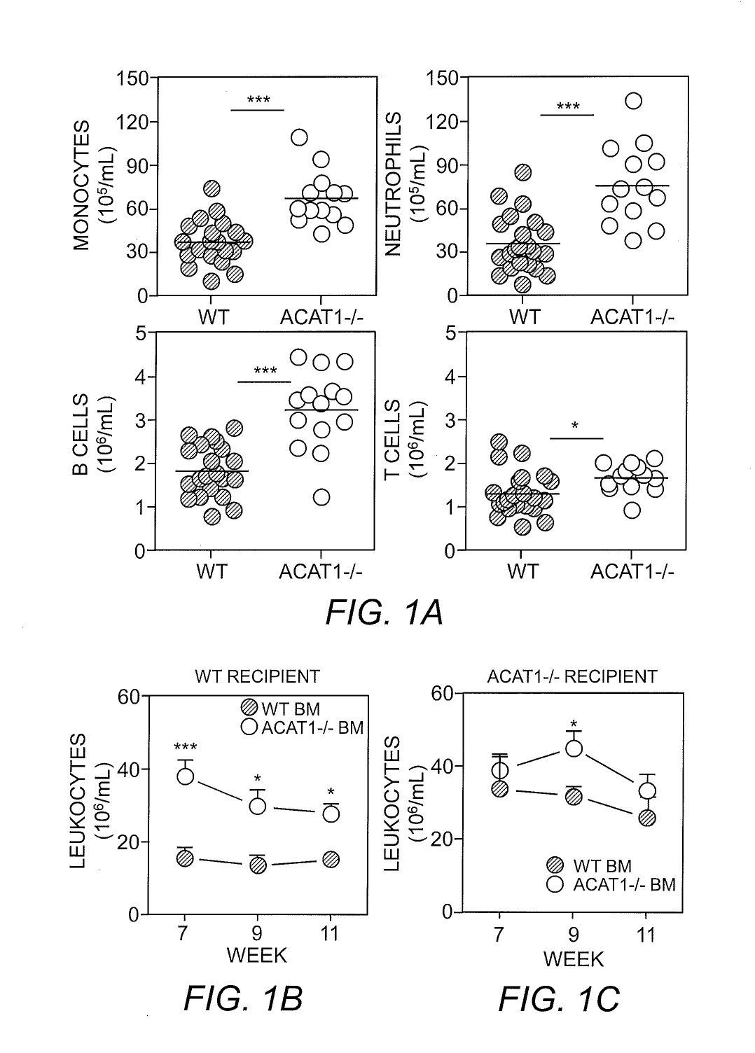

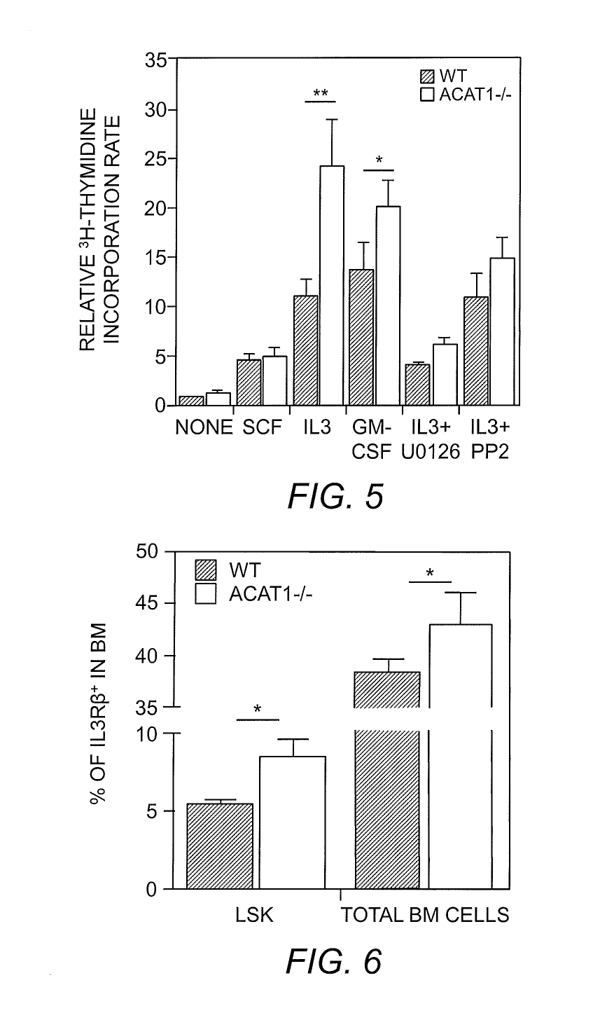

A physician having ordinary skill in the art can readily determine and prescribe the effective amount of the pharmaceutical composition required based upon the administration of similar compounds or experimental determination. For example, the physician could start doses of an agent at levels lower than that required in order to achieve the desired therapeutic effect and gradually increase the dosage until the desired effect is achieved. This is considered to be within the skill of the artisan and one can review the existing literature on a specific agent or similar agents to determine optimal dosing. By way of illustration, previous studies showed that in small animals, CI 1011 at 4-30 mg/kg/day is effective in reducing cholesterol ester content in various systemic tissues (Llaverias, et al. 2003. Cardiovascular Drug Reviews 21:33-50. In addition, administration of Avasimibe at a dose of 14.4 mg/kg/day has been shown to provide beneficial activity in a mouse model for Alzheimer's disease (Huttunen et al. 2009. FASEB J. 23:3819-3838). Avasimibe is administered to the normal mouse (i.e., the Acat1+/+ mouse) on a daily basis at different doses from 4 to 30 mg/kg/day. The effect of CI 1011 at different doses in causing leukocytosis will be monitored and compared with the leukocytosis phenomenon observed in the Acat1 KO mouse described in FIG. 1 and FIG. 5 of this invention.

The following non-limiting examples are provided to further illustrate the present invention.

### Example 1

### Materials and Methods

Animal Care.

Wild-type and the Acat1−/− mice (Meiner et al. 1996. Proc. Natl. Acad. Sci. USA 93:14041-14046) were maintained on a C57BL/6 background as described (Bryleva et al. 2010. Proc. Natl. Acad. Sci. USA 107:3081-3086). Protocols for the animal studies were approved by Dartmouth Animal Care Committee.

Cell Isolation and Counting.

Peripheral blood cells or bone marrow (BM) cells were isolated from age- and sex-matched mice using methods previously described (Jude et al. 2007 Cell Stem Cell. 1:324-337). Total cells were counted by using a Coulter cell counter. Blood cells or BM cells were incubated with red blood cell lysis buffer (e-Bioscience) for 15 minutes at ambient temperature. After washing once with phosphate-buffered saline (PBS) containing 3% FBS, cells were re-suspended in the same buffer. Leukocyte numbers were calculated after gating out red blood cells by using flow cytometry. Inguinal lymph nodes (LNs) were removed, homogenized, and cells were counted.

Flow Cytometry Analysis.

Cell populations were gated according to known methods (Yvan-Charvet et al. 2010. Science 328:1689-1693). Peripheral blood or BM leukocytes from age- and sex-matched mice were filtered using 40 micron cell strainers and stained with various specific antibodies for 15 minutes at 4° C. followed by a wash with PBS containing 3% FBS. For peripheral leukocytes, monocyte and neutrophil populations were quantified by staining with CD11b (1/70)-APC and with Gr1 (RB6-8C5)-PE; B and T populations were quantified by staining with CD3e (145-2C11)-PE and with B220 (RA3-6B2)-APC; antibodies were from Biolegend. For hematopoietic populations, the following lineage antibodies from Biolegend were used: c-Kit (2B8)-APC, Sca-1 (D7)-PE, CD34 (HM34)-Biotin, Streptavidin-APC-Cy7, Streptavidin-PE-Cy5, FcgRII/III (93)-FITC, and IL7Rα (A7R34)-FITC. Lineage antibodies were from a mouse lineage panel kit, p-ERK1/2-PE and CD131 (IL3Rβ) (JORO50)-PE, from BD Biosciences. Cells were analyzed on a BD FASCAN machine (BD Biosciences) installed with a 25 mW diode red laser and Rainbow software (Cytek development, Fremont, Calif.), or sorted on a BD FACSARIA Cell Sorter (BD Biosciences). Data were analyzed by using Flow Jo software from Tree Star, Inc.

Bone Marrow Transplantation.

The experiments were carried out as described (Jude et al. 2007. supra), using 8 week old female wild-type (WT) or Acat1−/− mice as recipients. After lysis of red blood cells, BM cells (2×10⁶) from each genotype were transplanted into lethally irradiated recipient WT mice or Acat1−/− mice through retro-orbital injection. Lethal irradiation was achieved using a Cs¹³⁷ irradiator using a split dose of 950 Rads.

Colony-Forming Assay.

The experiments were carried as described (Jude et al. 2007. supra). Twenty thousand BM cells isolated from 8 week old WT or Acat1−/− mice were cultured in methylcellulose media (MethoCult 3434; Stem Cell Technologies) at 37° C. Colonies were counted after 7 days.

Proliferation Assays.

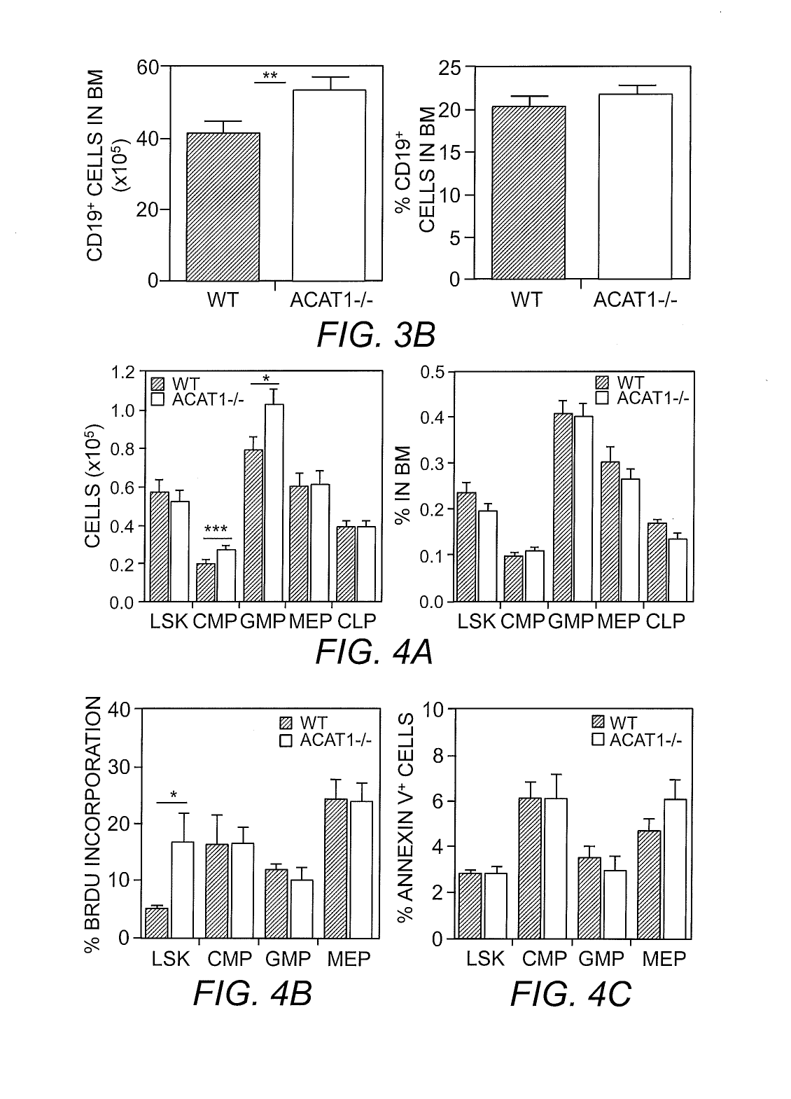

For cell proliferation measurement in vivo, the manufacturer's protocol (BD Pharmingen; BrdU flow kit) was used. Age- and sex-matched mice of both genotypes were injected intraperitoneally twice, once every 12 hours, with 0.5 mg bromodeoxyuridine (BrdU)/injection/mouse. Twenty-four hours after the first injection, BM progenitor cells (LSKs, CMPs, GMPs, and MEPs) were stained and sorted as described earlier. Proliferation of each of the sorted cell populations was measured by measuring BrdU incorporation, using the BrdU flow kit (BD Pharmingen). Proliferation measurement of BM derived-myeloid cells in culture was carried out using a known method (Yvan-Charvet et al. 2010. supra). Briefly, after lysis of red blood cells from crude BM, cells were cultured in IMDM (Gibco) containing 10% fetal bovine serum (FBS) for 2 hours to remove attached cells. The unattached cells were replated for 72 hours in various conditions as described in FIG. 4. Stem cell factor, IL3, and GM-CSF were from R&D Systems. U0126, PP2, and ApoA1 were from Sigma. Squalene synthase inhibitor (SSI) compound CP-340868 was from Pfizer. Cells were pulsed with 2 μCi/mL ³H-thymidine for 3 hours and ³H-thymidine incorporation was monitored by using liquid scintillation counter.

Annexin V Staining.

Apoptotic cell populations were measured using FITC-annexin V staining (BD Pharmingen). The staining procedure was according to the manufacturer's protocol. The analysis of annexin V positive cells was performed using a BD FASCAN machine (BD Biosciences).

Western Blot and RT-PCR Analyses.

To prepare BM derived-myeloid cells in culture for western blot analysis, cells were washed two times with PBS, cell pellets were lysed in RIPA buffer containing 0.15 M NaCl, 0.01 M Tris-Cl (pH 7.4), 1% NP-40, and protease inhibitors, incubated at room temperature for 30 minutes, followed by spinning at 14,000 rpm for 5 minutes to remove cell debris. Supernatant was collected and adjusted to 0.1 M DTT, 2% SDS and 1× sample buffer. Samples were resolved by SDS-PAGE and transferred to 0.22-μm nitrocellulose membranes. After blocking with non-fat milk dissolved in Tris buffer containing 0.1% TWEEN 20 (TBST) at room temperature for 30 minutes, the primary antibodies, p-ERK or ERK (Cell Signaling) in TBST were incubated with membrane, followed by incubation with secondary antibodies and detected by ECL chemiluminescence (Pierce). Acat1 gene and protein expressions in leukocytes were analyzed by western blotting and by RT-PCR according to established methods (Sakashita et al. 2003. Lab Invest. 83:1569-1581).

Statistical Analysis.

Results are reported as means±SEM. Two-tailed parametric student's t test and two-way ANOVA analysis with Bonferroni multiple comparison post-test were used to evaluate the statistical significance among various study groups. Analysis was performed and plotted by using GraphPad Prism 5.0. The difference between two sets of values was considered significant when the p value was <0.05 (*p<0.05, **p<0.01, ***p<0.001).

### Example 2

### Acat1 Knockout Mice Exhibit Leukocytosis

To test the hypothesis that global Acat1 loss may, affect hematopoiesis, the numbers of monocytes, neutrophils, B cells, and T cells in peripheral blood of age-matched WT and Acat1−/− mice were compared. The results showed that Acat1−/− mice contained significantly higher numbers of monocytes (CD11b⁺), neutrophils (Gr1⁺), and B cells (CD19⁺), in their blood compared to WT littermates (FIG. 1A). The Acat1−/− mice also contained slightly higher numbers of T cells than WT mice. In addition, their spleen size was bigger, and more cells were present in the inguinal lymph nodes compared to WT mice. The body weights of WT and Acat1−/− mice are the same, for both males and females.

The increase in leukocytes in Acat1−/− mouse could be caused by a cell-autonomous proliferation or survival difference of cells within BM, and/or by change(s) in tissue microenvironment in these mice. To address this issue, bone marrow transplantation (BMT) experiments were performed. BM from WT or from Acat1−/− mice were transplanted into lethally irradiated WT mice to produce chimeric mice. Seven weeks after transplantation, Acat1 gene expression in leukocytes isolated from the chimeric mice was tested by RT-PCR and by western blot analysis. The results confirmed that expression of both Acat1 mRNA and ACAT1 protein in mice transplanted with Acat1−/− BM was less than 10% of that of mice transplanted with Acat1+/+BM. Blood leukocyte numbers were also monitored in chimeric mice at different time points, from 7 week to 11 week after transplantation. The results showed that leukocyte numbers in mice transplanted with Acat1−/− BM were significantly higher than the value found in mice transplanted with WT BM (FIG. 1B). Parallel experiments were also performed by using lethally irradiated Acat1−/− mice as recipients of BM cells from either WT mice or Acat1−/− mice. Western blot and RT-PCR analyses showed that the expressions of both Acat1 mRNA and ACAT1 protein in mice transplanted with Acat1−/− BM were less than 10% of values found in mice transplanted with Acat1+/+ BM. Leukocyte number in chimeric mice was monitored, from 7 to 11 weeks after transplantation. The results showed that leukocyte numbers in Acat1−/− recipient mice (FIG. 1C) were higher than the values found in WT recipient mice (FIG. 1B), when donor BM came from either the WT mice (comparing filled circles in FIGS. 1B and 1C), or from the Acat1−/− mice (comparing open circles in FIGS. 1B and 1C). Additional results showed that the spleen weights of the chimeric mice with BM from the Acat1−/− mice were larger than those with BM from the WT mice. Together, these results indicated that Acat1 loss caused higher leukocyte numbers in the blood by affecting both cell autonomous proliferation in BM and by affecting the microenvironment supporting hematopoiesis. The rest of the work presented here focuses on the cell-intrinsic effects of Acat1−/− in hematopoietic cells.

### Example 3

### White Blood Cells of Acat1−/− Mice are Functional

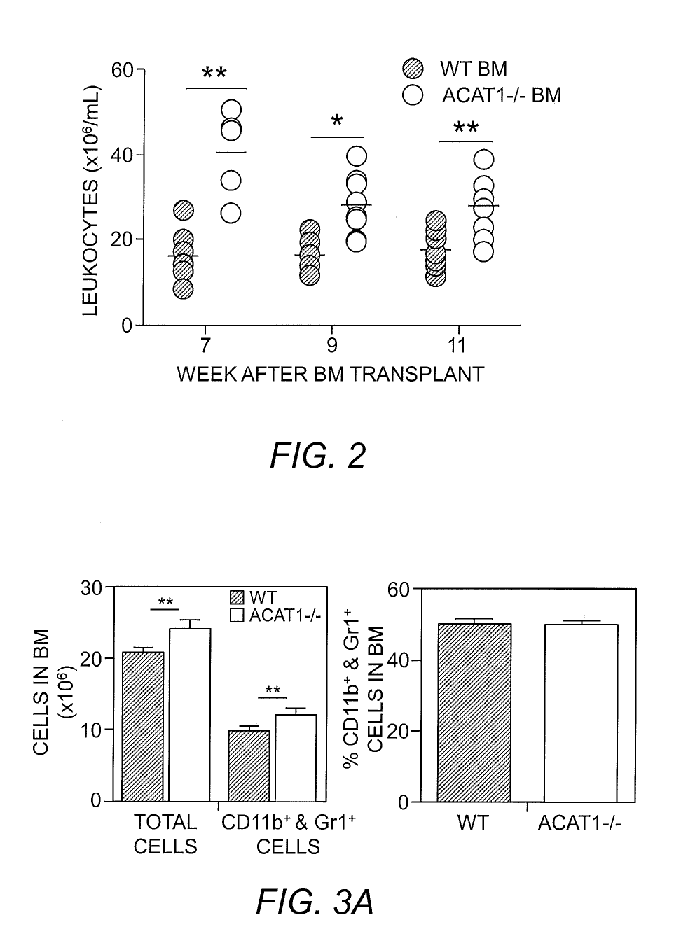

Although Acat1−/− mice have a higher number of white blood cells, it is important to demonstrate that the cells have functional activity. To test the functionality of leukocytes in Acat1−/− mice, a septic peritonitis assay was performed as has been previously described (LaFleur et al. 2004. Med. Inflamm. 13:349-355). The results demonstrated that 1-5 days after thioglycolate injection, more leukocytes are found in the peritoneum of mouse lacking Acat1 (Acat1−/− mice), demonstrating that leukocytes present in Acat1−/− are biologically active, and they migrate into the peritoneum in response to acute inflammation (FIG. 2).

The results of these experiments provide the basis for use of ACAT inhibitors as treatment for immunosuppression and to increase white blood cell counts in humans. As will be understood by one of skill in the art, results of drug testing in preclinical models of disease are routinely used to set doses for testing in humans. The effect of the ACAT inhibitors in humans can be monitored by measuring the white blood cells counts after treating the subjects with the inhibitors for various periods of time (i.e., one to several days). Doses for testing in humans can be determined by one of skill in the art. It is anticipated that the method of the present invention could involve administration of an ACAT inhibitor either alone or in combination with other drugs known to have efficacy in treating symptoms of immunosuppression. The ACAT1 inhibitor can be administered to a patient in a pharmaceutically acceptable vehicle by any desired route.

### Example 4

### Acat1 Loss Increases Hematopoietic Progenitor Cell Proliferation

To examine the effects of Acat1 loss on homeostasis of bone marrow cells, the number and the percentage of myeloid (Gr1⁺CD11b⁺) cell and B cell (CD19⁺) lineages in the BM, isolated from age matched (8 week old) WT and Acat1−/− mice, were compared. The results showed that Acat1−/− BM contained higher numbers of cells in the myeloid and the B cell lineages (FIG. 3A); while the percentages of these cells in WT and Acat1−/− BMs remained the same (FIG. 3D). These results indicated that Acat1−/− affected progenitor populations that differentiated into both myeloid cells and B cells. To test this possibility, the number and percentage of the following progenitor cells: LSK (Lin⁻Sca-1⁺c-Kit⁺), CMP (Lin⁻Sca-1⁻c-Kit⁺CD34⁺FcRII/III^{low}), GMP (Lin⁻Sca-1⁻c-Kit⁺CD34⁺FcRII/III⁺), MEP (Lin⁻Sca-1⁻c-Kit⁺CD34⁻FcRII/III⁻), and CLP (IL7α⁺Lin⁻Sca-1^{mid}c-Kit^{mid}) cells were compared in BMs isolated from the WT and the Acat1−/− mice. The results showed that the numbers of CMPs and GMPs in the Acat1−/− BM were increased, whereas the numbers of LSKs, MEPs, and CLPs in WT and Acat1−/− BMs were similar (FIG. 4A). These results indicated that Acat1−/− caused selective expansion of CMPs and GMPs progenitor populations and increased myeloid cell lineages in BM. The colony forming unit (CFU) assay in vitro has been used to estimate the frequency of primitive hematopoietic progenitor cells committed to the myeloid lineages. CFUs were enumerated and it was shown that Acat1−/− mouse BM produced significantly more colonies than the WT mouse BM. BrdU incorporation assays were next performed to monitor cell proliferation in WT and Acat1−/− mouse BMs in vivo. The results showed that when compared to WT BM, the LSK cells, but not other cell populations in Acat1−/− BM, exhibited a higher percentage of BrdU incorporation (FIG. 4B). This result supports the interpretation that increased numbers of CMPs, GMPs and myeloid cells observed in Acat1−/− BM (FIG. 4A) was mainly due to higher proliferation in Acat1−/− LSK cells. In parallel experiments, apoptotic cells in vivo were quantified in WT and Acat1−/− BMs by using the Annexin V staining assay. The results (FIG. 4C) showed that no significant difference in apoptotic cells could be detected between WT and Acat1−/− BM.

To confirm the finding that Acat1−/− enhances specific LSK cell proliferation at the in vitro level, various progenitor cells were isolated by cell sorting, including LSK, CMP, and GMP, from BMs of WT and Acat1−/− mice. The cytokine IL3 supports proliferation in several myeloid progenitor populations (Garland & Crompton. 1983. Exp. Hematol. 11:757-761 24). These purified cells were cultured in medium with 10% FBS supplemented with IL3 for 3 days then the BrdU incorporation assay was performed in vitro. The results showed that when compared with cells from the WT BM, only the LSK cells, but not the CMP or GMP cells from the Acat1−/− BM exhibited a significantly higher percentage of BrdU incorporation. These results were in accord with the in vivo data and demonstrated that Acat1−/−LSK cells proliferated more frequently in vivo and in vitro.

### Example 5

### Acat1−/− Increases BM Cell Proliferation by Activating the ERK Pathway

It has been shown that upon stimulation by IL3, BMs isolated from an ABCA1/ABCG1 double KO mouse exhibited increased LSK cell proliferation in vitro, mainly through activated p-ERK Pathway (Yvan-Charvet et al. 2010. Science 328:1689-1693). Aberrant ERK signaling may also mediate the effect of Acat1−/− on LSK cell proliferation. To test the possibility, WT and Acat1−/− BM cells were first cultured in 10% FBS supplemented with various cytokines (SCF, or IL3, or GM-CSF as indicated). Proliferation rates of the cells were then monitored using a ³H-thymidine pulse. The results (FIG. 5) showed that both the IL3-treated and the GM-CSF-treated Acat1−/− BM cells, but not the SCF-treated Acat1−/− BM cells, had significantly higher cell proliferation then their WT counterparts (FIG. 5A). LSK, CMP, and GMP cells were next purified from the WT and the Acat1−/− BMs and it was shown that IL3 treatment also caused purified Acat1−/− LSK cells to incorporate more BrdU than their WT counterparts. These results showed that the IL3-dependent BM cell proliferation in vitro was enhanced by Acat1 loss. The degree of p-ERK activation in IL3-treated WT and Acat1−/− BM cells was next compared. The results of a western blot analysis showed that in response to IL3 treatment, Acat1−/− cells exhibited enhanced p-ERK than WT cells. To validate the results shown in FIG. 4A, a different approach was took using flow cytometry to monitor the p-ERK signal within CD11b-positive cells after IL3 treatment. The results showed that the Acat1−/− BM cell culture exhibited higher p-ERK within the Cd11b-positive cells than the WT counterpart (30% vs 13%). WT and Acat1−/− BM cell cultures were subsequently treated with two different specific ERK signaling pathway inhibitors, MEK inhibitor (U0126) and Src tyrosine kinase inhibitor (PP2) (McCubrey et al. 2006. Adv. Enzyme Regul. 26:249-279; Chang et al. 2003. Leukemia 17:1263-1293). The results showed that both inhibitors partially alleviated the higher ³H-thymidine incorporation phenotype observed in the Acat1−/− BM cells (FIG. 5), supporting the conclusion that Acat1−/− enhanced ERK signaling pathway in BM myeloid cells. The IL3Rβ subunit is shared by the IL3 and GM-CSF receptors, and plays a critical role in receptor mediated ERK signaling pathway (Yvan-Charvet et al. 2010. supra; Chang et al. 2003. supra). Cell surface IL3Rβ expression was assessed and it was found that in Acat1−/− BM, the IL3Rβ signal was increased in LSK cells, and in the bulk BM cells in vivo (FIG. 6). In addition, it was found that the cell surface IL3Rβ expression was increased in Acat1−/− BM cell culture under IL3 treatment in vitro, when compared to its WT counterpart (42% vs 60%). Taken together, these results support the conclusion that Acat1−/− LSK cells exhibit enhanced IL3-dependent proliferation due to increased IL3Rβ expression and thus enhanced ERK signaling. Interestingly, increased cell surface IL3Rβ expression was also observed in BMs lacking both ABCA1 and ABCG1 (Yvan-Charvet et al. 2010. supra).

ACAT1 converts cholesterol to cholesteryl esters, and prevents the accumulation of free cholesterol in various cell membranes (Chang et al. 2009. Am. J. Physiol. Endocrinol. Metab. 297:E1-9). IL3Rβ is mainly expressed at the plasma membrane (Chang et al. 2003. supra). Signaling of IL3 through IL3Rβ is affected by cellular membrane lipid composition. The effect of Acat1−/− on IL3 signaling may be attributed to a change in the cholesterol content in BM cells. To test this possibility, a small molecule squalene synthase inhibitor (SSI) CP-340868, which blocks the biosynthesis of squalene from farnesyl pyrophosphate, was employed. At micromolar concentrations, this inhibitor effectively shuts down cholesterol biosynthesis without inhibition of non-sterol polyisoprenoid biosynthesis and protein prenylation (Harwood et al. 1997. Biochem. Pharmacol. 53:839-864; Pandit et al. 2000. J. Biol. Chem. 275:30610-30617). WT and Acat1−/− BM cells were incubated with IL3 in the presence or absence of CP-340868, and it was found that treating cells with CP-340868 reversed the enhanced cell proliferation observed in Acat1−/− BM cells. The result of a parallel experiment showed that adding ApoA1 (at 50 or 100 μg/ml), a cellular cholesterol efflux mediator, to the cell culture did not diminish the enhanced proliferation observed in Acat1−/− BM cells. These results indicated that de novo cholesterol synthesis in BM cells was required to mediate the effect of Acat1−/− on cell proliferation.

### Example 6

### Acat1−/− Animals Exhibit an Increase in B Cell Progenitors

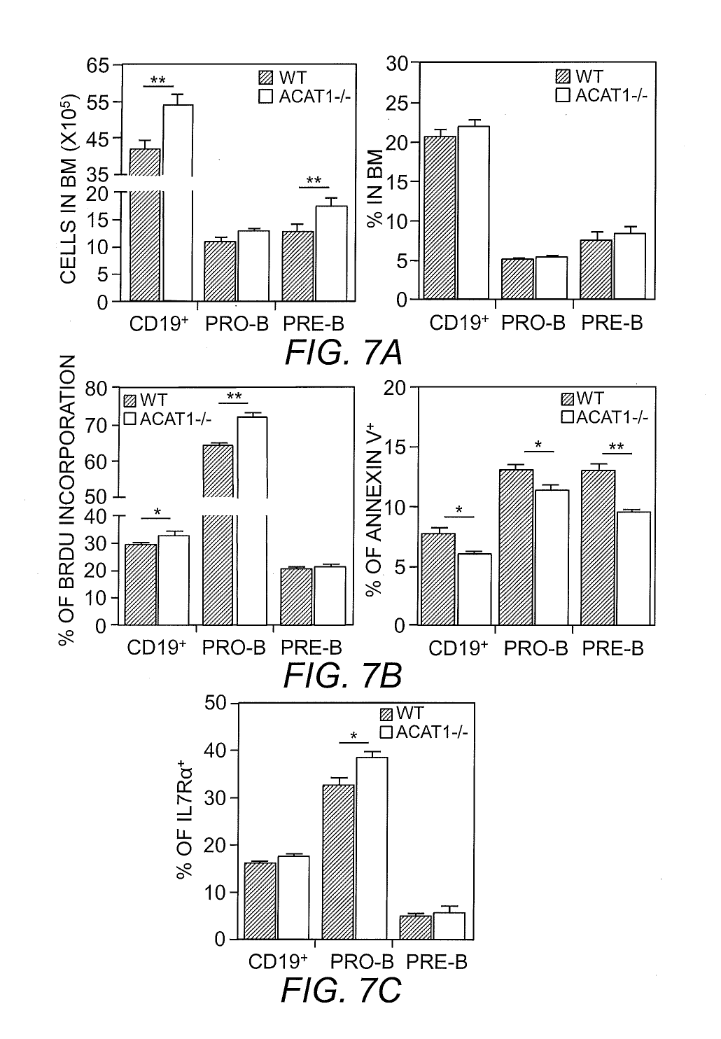

It was shown that in Acat1−/− mouse blood, in addition to the myeloid cell number, the B cell number (CD19+) was also increased (FIG. 1A). The common lymphoid progenitor (CLP) is the major progenitor for B lineage-restricted cells. However, no difference in number and in percentage of CLPs could be found between the WT and the Acat1−/− BMs (FIG. 4A). These results implied that Acat1 loss may affect B cell progenitor populations downstream of CLPs. To test this possibility, specific B cell progenitor populations were monitored, and it was found that when compared to WT, the Pre-B cell population, but not the Pro-B cell population, was increased in Acat1−/− BM (FIG. 7A; left panel). The percentage of B cell progenitors in WT and Acat1−/− BMs were the same (FIG. 7A; right panel). It was next found that the BrdU incorporation in vivo was higher in Acat1−/− Pro-B cells than their WT counterpart (FIG. 7B; left panel). The result of a parallel experiment showed that apoptotic cells in vivo (as quantified by Annexin V staining) were reduced in Acat1−/− Pro-B cell population when compared to the WT counterpart (FIG. 7B; right panel). In B cell progenitors, the IL7 receptor a subunit plays a key role both in survival and in proliferation (Nagasawa. 2006. Nat. Rev. Immunol. 6:107-116). The expression of cell surface IL7Rα in bone marrow B cells was compared, and it was found that its expression was elevated in Acat1−/− Pro-B cells when compared to WT Pro-B cells (FIG. 7C). This result indicated that Acat1 loss facilitated proliferation of Pro-B cells in part through elevated cell surface IL7Rα expression, similar to the findings with the IL3Rβ in the myeloid lineage.

### Example 7

### Acat1−M/−M Mouse Model

For the purpose of studying the function of ACAT1 in monocytes/macrophages specifically, the homozygous conditional Acat1 mouse (Acat1flox/flox mouse) was created (by using an Acat1/loxP construct that contains Acat1 exon 14, which includes the key catalytic residue His450 flanked by the loxP sites). The Acat1flox/flox mouse was crossed with the mouse expressing the Cre recombinase transgene under control of the lysozyme M promoter (LysCre+/+) to obtain a mouse with macrophage-specific deletion of Acat1 (Acat1−M/−M). To demonstrate the characteristics of the Acat1−M/−M mice, peritoneal macrophages were isolated from age-matched (7- to 8-week-old) wild-type and Acat1−M/−M mice and their ACAT1 mRNA levels, ACAT1 protein contents, and cholesteryl ester biosynthesis rates were compared using methods previously described (Sakashita et al. 2003. Lab. Invest. 83:1569-1581; Sakashita et al. 2010. J. Lipid Res. 51:1263-1272). Results showed that the Acat1 mRNA levels, ACAT1 protein levels, and cholesterol esterification activity in intact cells in macrophages from the Acat1−M/−M mice were all decreased by more than 80% when compared with the wild-type mice. In another experiment, it was found that the ACAT1 protein content in adrenal glands from the Acat1−M/−M mouse was normal, demonstrating the specificity of Acat1−M/−M mouse phenotype in terms of Acat1 expression knockout being limited to macrophages.

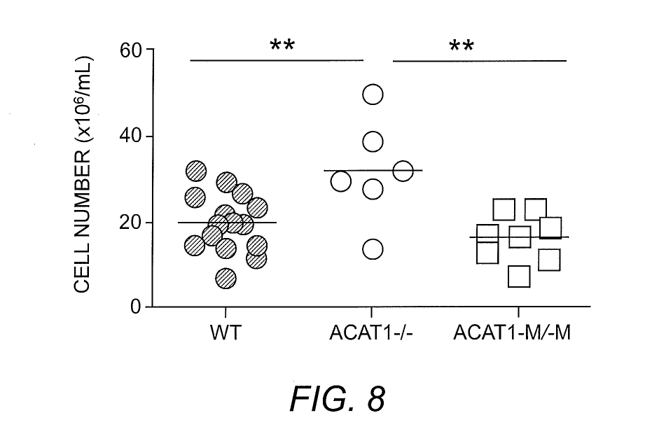

This new mouse model was then used as a control to examine the effect of Acat1 inhibition on white blood cell count. Using the Acat1−M/−M mouse as a control, it was found that, in mice that were 7 to 8 weeks of age (age- and sex-matched), in contrast to total knockout of Acat1 in Acat1−/− mice, the Acat1−M/−M mice do not contain increased numbers of white blood cells in blood (FIG. 8 [5]). The white blood cell populations were analyzed using flow cytometry. This result demonstrates that the effect of Acat1−/− in increasing the white blood cells occur in hematopoietic stem cells, not in monocytes/macrophages.

### Example 8

### siRNA Inhibition of ACAT1

Four different siRNA sequences (#52-#55) targeting the mouse Acat1 (also called Soat1) gene were inserted into an endogenous mouse microRNA (miR) scaffold using Invitrogen's RNAi design tool. The artificial miRs were ligated into the mammalian expression vector pcDNA6.2-GW/EmGFP-miR. These Acat1miR constructs were tested along with a negative control (NC) miR (5′-TACTGCGCGTGGAGACG-3′; SEQ ID NO:9), which does not match the sequence of any known vertebrate gene, in NIH-3T3 mouse fibroblasts. The miRs were delivered to the cells by using standard cDNA transfection protocol. The results show that two of the Acat1 miRs (containing the siRNA sequence #54, 5′-TACAGTAGGAGTCCTTGGGTA-3′, SEQ ID NO:10; and sequence #55, 5′-TCCAGTATCAGAATGAACCGGG-3′, SEQ ID NO:11) were effective in causing a 50-60% reduction in the ACAT1 protein content in treated mouse 3T3 fibroblasts. These two Acat1 miR sequences and the NC miR sequence were also subcloned into an rAAV backbone vector (AAV-6P-SEWB) that contained the neuron-specific hSyn promoter. This vector contains a strong and cell-type-nonspecific promoter that expresses Acat1 miRs in any cell type where the viral genome is expressed. For identification purpose, it also coexpresses the GFP with the miRs. These three constructs were used to produce three recombinant AAV viruses. To test the efficacy and specificity of these viruses, cultured primary hippocampal neurons were treated with the NC AAV, or with AAV that expresses miR that contain siRNA Acat1 #55. Two weeks after viral infection, the effects of AAVs on cholesteryl ester biosynthesis in neurons were tested. The results showed that the AAV harboring siRNA Acat1 #55 reduced cholesteryl ester biosynthesis by more than 50%, when compared with values in NC virus treated cells. Subsequently, the NC AAV or the Acat1 AAV (that include both siRNA Acat1 #54 and #55) were injected into the hippocampal region of mice at 4 month of age. After a single bilateral injection, mice were allowed to recover. One month after injection, mice were sacrificed and the ACAT1 enzyme activities in the mouse brain homogenates were analyzed by using standard ACAT enzyme activity assay in vitro. The result shows that when compared with the control values, the Acat1 AAV reduced ACAT1 enzyme activity by 42%.

1. A method for treating immunosuppression comprising administering to a patient diagnosed with low white blood cell count an effective amount of an Acyl-coenzyme A:cholesterol acyltransferase (ACAT) inhibitor in a pharmaceutically acceptable vehicle, so that the signs or symptoms of low white blood cell count are decreased, delayed, or prevented thereby treating immunosuppression.

2. The method of claim 1, wherein said ACAT inhibitor inhibits both ACAT1 and ACAT2.

3. The method of claim 1, wherein said ACAT inhibitor is a selective inhibitor of ACAT1.

4. The method of claim 3, wherein the ACAT inhibitor has an IC₅₀ value for ACAT1 which is at least twice the corresponding IC₅₀ value for ACAT2.

5. The method of 3, wherein the selective inhibitor of ACAT1 is an siRNA or microRNA.

6. The method of claim 1, wherein the ACAT inhibitor is administered via a liposome or nanoparticle.

7. The method of claim 1, wherein the ACAT inhibitor has an IC₅₀ value in the range of 1 nM to 100 μM.

8. The method of claim 1, wherein the ACAT inhibitor is administered in combination another drug used to treat immunosuppression.


SEQUENCE LISTING (11 sequences)

SEQ ID NO: 1 - DNA, 21 bp, Artificial Sequence
  Synthetic oligonucleotide
ggagctgaagccactatttat

SEQ ID NO: 2 - DNA, 22 bp, Artificial Sequence
  Synthetic oligonucleotide
ctgtttgaagtggaccacatca

SEQ ID NO: 3 - DNA, 22 bp, Artificial Sequence
  Synthetic oligonucleotide
cccggttcattctgatactgga

SEQ ID NO: 4 - DNA, 24 bp, Artificial Sequence
  Synthetic oligonucleotide
aactacccaaggactcctactgta

SEQ ID NO: 5 - DNA, 64 bp, Artificial Sequence
  Synthetic oligonucleotide
tgctgtccagtatcagaatgaaccgggttttggccactgactgacccggttcactgatactgga

SEQ ID NO: 6 - DNA, 64 bp, Artificial Sequence
  Synthetic oligonucleotide
tgctgtacagtaggagtccttgggtagttttggccactgactgactacccaagctcctactgta

SEQ ID NO: 7 - RNA, 25 nt, Artificial sequence
  Synthetic oligonucleotide
caugaucuuccagauuggaguucua

SEQ ID NO: 8 - RNA, 25 nt, Artificial sequence
  Synthetic oligonucleotide
uagaacuccaaucuggaagaucaug

SEQ ID NO: 9 - DNA, 17 bp, Artificial sequence
  Synthetic oligonucleotide
tactgcgcgtggagacg

SEQ ID NO: 10 - DNA, 21 bp, Artificial sequence
  Synthetic oligonucleotide
tacagtaggagtccttgggta

SEQ ID NO: 11 - DNA, 22 bp, Artificial sequence
  Synthetic oligonucleotide
tccagtatcagaatgaaccggg